(19) United States
(12) Patent Application Publication
(10) Pub. No.: US 2016/0158410 A1
(43) Pub. Date: Jun. 9, 2016

(54) POLYSACCHARIDE HYDROGELS FOR INJECTION WITH TUNABLE PROPERTIES

(71) Applicants: ALBERT-LUDWIGS-UNIVERSITÄT FREIBURG, Freiburg (DE); UNIVERSITÄTSSPITAL BASEL, Basel (CH)

(72) Inventors: Prasad V. SHASTRI, Freiburg (DE); Andrea BANFI, Basel (CH); Aurelien FORGET, Freiburg (DE); Roberto GIANNI-BARRERA, Maennedorf (CH)

(21) Appl. No.: 14/906,806

(22) PCT Filed: Jul. 21, 2014
(86) PCT No.: PCT/EP2014/065649

§ 371 Date: Jan. 21, 2016

(30) Foreign Application Priority Data
Jul. 22, 2013 (EP) 13177505.8

(51) Int. Cl. A61L 27/20; A61L 27/36; A61L 27/54; A61L 27/52; A61L 27/22

(52) U.S. Cl. CPC A61L 27/20; A61L 27/52; A61L 27/22; A61L 27/54; A61L 27/3633; A61L 2400/06; A61L 2300/414

(57) ABSTRACT

Injectable hydrogels comprising polysaccharides based on disaccharides the backbones of which form an α-helix structure and in which in at least 10% of the disaccharide units the primary hydroxyl groups are oxidized.

The present invention relates to polysaccharide hydrogels with tunable properties like stiffness and provasculogenic properties.

The organization of cells into tissue-like structures involves a complex interplay between soluble signals and those originating from the extracellular matrix (ECM).

In recent years several studies have shown that cells can respond to physical cues (substrate stiffness and nanoroughness) in a very well-defined manner, and this may constitute a new form of signaling (1-3). Mechanobiology—the interplay between biological and physical signals in establishing cell function—constitutes a new avenue for deciphering the signaling environment during tissue morphogenesis. In this regard, there is a need to develop systems that can enable the investigation and translation of mechanobiology paradigms into regenerative medicine solutions in vivo (4-6).

Such a system should meet the following criteria: offer precise tailoring of the mechanical environment in vivo, be cytocompatible, enable predictable evolution of cellular function, and exhibit human biocompatibility.

Hydrogels, by virtue of their ability to mimic several aspects of physiological environments such as hydration state and interconnected pore architecture, have been explored extensively in this context as mimics of extracellular matrices (ECM (4)) and in the de novo development of tissue (7-9).

Hydrogels can be formed from either synthetic or natural water-soluble polymers, and the transformation of a polymer network into a gel requires the introduction of cross-links (net points) between polymer chains. Hydrogels of polyethylene glycol (PEG) and hyaluronic acid (HA), an ECM component, constitute the most prominent class of hydrogels for regenerative medicine applications, and they are formed through radical photopolymerization (10), Michael addition (vinyl sulfone) (11), click chemistry (thiolene) (12)] or enzymatic (trans-glutaminase) cross-linking (13). In addition to HA, other polysaccharides such as alginate (14), which undergoes calcium-induced gelation (15, 16), and chitin (17) have also been explored. More recently, self-assembled peptides have emerged as yet another class of biologically derived hydrogels (18, 19).

To use hydrogels as instructive materials in the context of mechanobiology, precise control over the mechanics and biology within the hydrogel is desirable.

In a chemically cross-linked system varying the modulus necessitates changing the polymer concentration and/or polymer chain length. Additionally, implementation of chemical cross-linking in vivo can be challenging as it requires initiators and chemistries, which can also react with ECM components and proteins, and when not consumed can lead to toxicity.

Agarose, a polysaccharide extracted from marine red algae composed of D-galactose-3,6-anhydro-L-galactopyranose repeat units, has received considerable attention in regenerative medicine in recent years due to its cytocompatibility, tissue compatibility in humans (20, 21), and ability to induce, in vivo, the de novo formation of hyaline-like cartilage (8) and is currently undergoing phase-3 clinical trials in humans as a carrier for chondrocytes (22). Unlike PEG, HA, and alginate, agarose forms a hydrogel through physical cross-linking (23), which in comparison with chemical and ionic cross-linking offers several advantages including the absence of reactive chemistry and ease of implementation.

Vascularization is an important biological process that is necessary for the development, repair and sustenance of tissue in mammals. Vascularization is the formation of new blood vessels from existing blood vessels through sprouting (angiogenesis) or the de novo organization of endothelial progenitor cells into vascular structures (vasculogenesis).

The constriction, or damage or loss of vasculature can lead to irreversible damage to tissue and is the cause of many pathologies and clinical conditions. For example, the constriction of coronary arteries (vessels that supply oxygenated blood to the heart muscle), which results in the formation of local ischemia (loss of blood supply) will lead to damage to the heart tissue resulting in a myocardial infarct.

Diabetes mellitus, a systemic disorder can lead to peripheral vascular disease resulting in loss of function in the extremities such as limbs due to ischemia and associated avascular necrosis. Constriction or damage to vasculature in the limbs can promote muscle degeneration.

In all of the aforementioned pathologies, the induction of new blood vessels (angiogenesis or vasculogenesis) is considered to be an important step in stabilizing or reversing the negative effects of ischemia.

The deliberate induction of new vasculature in a tissue through an external intervention is called Therapeutic Angiogenesis (TA). The goal of therapeutic angiogenesis is to stimulate the creation of new blood vessels in ischemic organs, tissues or parts with the goal to increase the level of oxygen-rich blood reaching these areas

Formation of new blood vessels can be triggered by the local administration of proteins such as vascular endothelial growth factor (VEGF), fibroblast growth factor (FGF), genetic material (plasmid or viral vectors) that encode for VEGF or FGF and/or endothelial progenitor cells (EPCs) with or without association with an injectable carrier. These efforts have largely resulted in the induction of vasculature that lacks appropriate physiological structural and functional traits and tends to regress over a period of time.

### BRIEF DESCRIPTION OF THE FIGURES

FIG. 1A shows the circular dichroism (CD) spectrum of a 0.15% wt/vol solution of natural agarose (NA) and 93% carboxylated agarose (93-CA) obtained below the gelation temperature.

FIG. 1B shows the plot of the ellipticity at 203 nm as a function of the degree of carboxylation;

FIG. 1C shows a Ramachandran plot for natural agarose (NA) and 93% carboxylated agarose (CA);

FIG. 2A shows the tapping mode atomic force microscopy (AFM) of single molecule height (main plot) and phase (Inset) for natural agarose and carboxylated agarose with various degrees of carboxylation;

FIG. 2B shows the Environmental Scanning Electron Microscopy (ESEM) of freeze dried 2% wt/vol hydrogel of natural agarose and carboxylated agarose with various degrees of carboxylation.

FIG. 2C shows the dependence of the gelation temperature of agarose hydrogels as a function of the degree of carboxylation;

FIG. 2D shows the CD spectrum of a 0.15% wt/vol solution of 93% carboxyl modified agarose (93-CA) below the gelation temperature (5° C.) and above the gelation temperature (90° C.)

FIG. 3A shows the comparison of the rheological behaviour of natural agarose and 60% carboxylated agarose (60 CA) through comparison of shear modulus (G′) and loss modulus (G″);

FIG. 3 B shows the shear modulus as a function of the degree of carboxylation at various hydrogel concentrations;

FIG. 4A shows the organization of human umbilical vein endothelial cells (HUVECs) into 2D lumens (L) in four types: oval structures composed of a single cell, type I; elliptical structures composed of two cells, type II; circular structures composed of two to four cells, type III; and circular structures composed of more than four cells, type IV. (Scale bar: 10 μm.) The color-coded bars at the bottom span morphologies typically observed under the various conditions.

FIG. 4B shows a scatter plot of diameter and cell numbers associated with lumens.

FIG. 4C shows large-scale organization of HUVECs.

FIGS. 4D-F show the apical-basal polarization of HUVECs in CA60 gels.

FIG. 4G shows the mRNA expression level of key provasculogenic markers in HUVECs. The expression of (from left to right for each of the four samples) PODXL, LAM5, CCM1, NID2 and COLIV in HUVECs in CA60 gels (Scale bar: 10 μm).

It was an object of the present invention to provide hydrogels, the physicochemical properties of which may be tuned over a wide range of properties. These hydrogels preferably should be suitable for use in therapeutic angiogenesis as described above.

This object has been achieved by injectable hydrogels comprising polysaccharides based on disaccharides the backbones of which form an α-helix structure and in which in at least 10% of the disaccharide units of the primary hydroxyl groups are oxidized.

The term hydrogel, as used herein, is intended to denote a water insoluble network of polymer chains in which water is the dispersion medium. Hydrogels possess a degree of flexibility similar to natural tissues.

Hydrogels are three-dimensional networks composed of hydrophilic polymers crosslinked either through covalent bonds or held together via physical intramolecular and/or intermolecular attractions.

Hydrogels differ from normal gels in a number of properties. Whereas gels are semi-solid materials made of hydrophilic polymers comprising small amounts of solids dispersed in relatively large amounts of liquid, hydrogels are also made up of hydrophilic polymer chains, but these chains are crosslinked. This enables hydrogels to swell while retaining their three dimensional structure without dissolving. Thus, the principle feature of hydrogels differentiating them from gels is their inherent crosslinking.

The injectable hydrogels in accordance with the present invention comprise polysaccharides based on disaccharides.

In a preferred embodiment the polysaccharide is derived from agarose. Agar, a structural polysaccharide of the cell walls of a variety of red seaweed, consists of two groups of polysaccharides, namely agarose and agaropectin. Agarose is a neutral, linear polysaccharide with no branching and has a backbone consisting of 1,3-linked β-D-galactose-(1-4)-α-L-3,6 anhydrogalactose repeating units. This dimeric repeating unit, called agarobiose differs from a similar dimeric repeating unit called carrabiose which is derived from carrageenan in that it contains 3,6-anhydrogalactose in the L-form and does not contain sulfate groups.

Other polysaccharides which may be mentioned here are hyaluronic acid, heparan sulfate, dermatan sulfate, chondroitin sulfate, alginate, chitosan, pullulan and κ-carrageenan.

Preferred examples of polysaccharides the backbones of which form an α-helix structure are agarose and ε-carrageenan.

The degree of oxidation of the primary hydroxyl groups may vary over a wide range and is at least 10%, preferably at least 11%, more preferably at least 20% and most preferably 35% or more. In some cases degrees of oxidation of from 50% to 95%, preferably of from 55 to 93% have shown to be advantageous. While it is in principle possible to completely oxidize the primary hydroxyl groups, degrees of modification of at maximum 99%, preferably at maximum 95% and even more preferably at max. 93% are preferred.

In certain cases oxidation of from 20 to 70%, preferably of from 25 to 60% has proved to be advantageous.

The percentages for the degree of modification are in per cent of the number of the respective groups in the polysaccharide.

Preferably the primary hydroxyl groups are oxidized into carboxylic acid groups.

The oxidation of primary hydroxyl groups in saccharide units can be effected in a variety of ways which are known to the skilled person, who will select the appropriate process based on his or her professional experience and suitable for the specific needs of the individual application.

Just by way of example, the oxidation with the well known oxidizing agent TEMPO ((2,2,6,6-Tetramethyl-piperidin-1-yl)oxyl), reactivated with NaOCl and catalyzed by potassium bromide may be mentioned here. Sodium hydroxide may be added during the reaction to maintain the optimum pH and to compensate the acidification of the solution due to the formation of the carboxylic acid groups. NaOH not only stabilizes the pH but also provides a quantitative measurement of the degree of oxidation as it compensates the carboxylic acid groups formed.

A possible side reaction, the conversion of the carboxylic acid group formed into an aldehyde group can be compensated by the addition of a reducing agent such as sodium borohydride (NaBH₄) which reduces the aldehyde formed back to the primary alcohol which can then be oxidized into the carboxyl group again.

The formation of carboxylic acid along the polysaccharide backbone can be monitored by the amount of NaOH during the reaction and afterwards by the quantitative analysis using FTIR spectrometry.

In order to determine as precisely as possible the percentage of the oxidized primary alcohol groups it is possible either to perform the oxidation reaction in a controlled manner or alternatively the polysaccharide is oxidized completely so that about 100% of the primary alcohol groups are oxidized. Such completely oxidized polysaccharide can be blended with unmodified polysaccharide which may either be the same polysaccharide or another polysaccharide. Thus, the chemical modification can be precisely controlled during the reaction or by controlling the blending with another polysaccharide or the same unmodified polysaccharide.

In accordance with a preferred embodiment of the present invention, the polysaccharide in the hydrogel is covalently modified with cell adhesion motifs such as the integrin binding sequence arginine-glycine-aspartic acid (RGD, which may occur in various alternatives, e.g. cyclic RGD, or variants), or with peptide sequences like YIGSR, IKVAV. MNYYSNS or PHSRN to name just a few examples.

In accordance with another preferred embodiment, the hydrogels in accordance with the present invention contain soluble signals such as e.g. vascular endothelial growth factor (VEGF), phorbol 12 myristate acetate (PMA), fibroblast growth factors (FGF), insulin growth factors (IGF), transforming growth factor beta-1(TGF-β) or platelet derived growth factor (PDGF).

The hydrogels in accordance with the present invention in accordance with another embodiment comprise components of the extracellular matrix (ECM) e.g. basement membrane proteins (BMP) such as collagen type 4 (Col4), laminins (LAM) or entactin (also known as nidogen) or mixtures thereof.

The BMPs can be introduced into the hydrogel using e.g. Matrigel, a gelatinous protein mixture commercially available from various sources.

BMP mimicking peptide sequences or BMP's extracted from other mammalian tissue than Matrigel may also be mentioned. The skilled person will select the appropriate system based on the individual needs of the specific application.

Polysaccharides whose backbones are organized into a-helices such as agarose and κ-carrageenan can undergo thermally reversible gelation from aqueous solutions. The key step in their physical gelation is the aggregation of the double-stranded α-helices (24, 25). It has been reported that oxidation of the D-galactose primary alcohol residue in agarose results in weaker gels (26).

Introduction of charged moieties such as carboxylic acid groups alters helical interactions and thus the gelation behaviour. Carboxylation of the primary hydroxyl group is an example for this effect.

The formation of double-stranded helices in proteins and oligonucleotides is driven by the ability of the macromolecular chains to form weak interactions through H bonds. It is therefore reasonable to assume that the potential for the formation of such interactions would be a function of the distance between the H atoms and the electronegative oxygen (alcohol or carboxylic acid). Carboxylation promotes separation of the polymer chains, which diminishes the likelihood of H-bonding interactions. The analysis of the frequency of H bonds on a per frame basis shows that carboxylation indeed decreases the propensity of at least one H-bond formation by over 75% compared with unmodified agarose. These observations taken in sum suggest the possibility that the introduction of a charged carboxylic acid group can potentially alter the associative behavior of the agarose helices.

As has been found in the course of the present invention, carboxylation promotes a β-sheet secondary structure. One potential outcome of introducing charges along a polymer backbone is a transition of the polymer chains from a coiled morphology to a more extended morphology due to increased electrostatic repulsion between the chains (27).

In the following the results of various investigations are described showing the influence of a partial oxidation of the primary hydroxyl groups with agarose as preferred polysaccharide. The results apply in an equal manner to other polysaccharides which have backbones forming an α-helix structure.

The analytical methods described below were used as follows:

### Circular Dichroism

Circular dichroism spectra were obtained using a Jasco spectropolarimeter

J-810 equipped with a Peltier temperature cell Jasco PFD-425S. Solution of 0.15% w/v of agarose was made in Milli-Q water at 90° C. for 15 min then the solution was cooled down at 5° C. in the CD chamber for 30 min prior to measurement. Each spectrum has been recorded three times and summed together. Each spectrum for a given modification is a mean of three different syntheses.

### Zeta Potential

Zeta potential has been measured on a Beckman Coulter Delsa Nano C particle analyzer. The same solutions have been used as for the light scattering experiment. Measurements have been made in a flow cell that has been aligned with the laser prior to every measurement. Each measurement has been made three times and an average has been calculated, each spectrum for a given modification is a mean of three different syntheses.

### ESEM

SEM pictures were obtain with a ref agarose gels of 2% w/v were prepared and 2 ml of this solution was frozen dried for 24 hours under 0.1 mbar vacuum in a 5 ml glass vial. The sample has then been vertically cut and the inside of the sample has been imaged at different magnification. Images shown here are representative of different areas of a given sample at different magnification, which have been reproduced with three different gels prepared from different batches.

### AFM

AFM pictures were obtained with a Veeco Dimension 2100. Samples were prepared on a 3 mm microscopic glass holder that had been passivated. The glass slide was washed with 0.1 M NaOH and dried in an oven. The dry slides were then passivated with few drops of dichloromethylsilane. Two slides were sandwiched together to have a uniform passivation. After 10 min the slides were washed with water and the excess of dichloromethylsilane was washed with soap and the slides were dried. Slides side was prepared in a hydrophobic way. Agarose samples were prepared as 2% w/v gels and 25 μl of the solution was poured onto an unmodified glass slide, a dichloromethylsilane passivated slide was then adjusted on top of the solution. Slides of 0.5 mm were put as spacer between the hydrophobic and the normal glass slide, the whole montage was then allowed to gel for 30 min at 4° C. The upper slide (hydrophobic) was removed thereafter and a thin layer of agarose gel was obtained. This gel was then allowed to stabilize at room temperature for 30 min before measurement in order to avoid any shrinkage or dilatation of the gel during the measurement.

### Molecular Dynamics (MD)

MD simulations have been done using the Desmond package of the Maestro version 8.5 from Schrodinger. Initial conformation has been obtained from the x-ray structure of the agarose that has been downloaded from the PDB library. Modified agarose has been drawn from the PDB file directly inside the Maestro software. Implicit water model has been build using the Desmond tool, resulting in a 10 Å square box build by following the TIP3 solution model. The simulations have been run in the model NPV at 300° K. at atmospheric pressure for 15 ns. Analysis of the results was done using the VMD software and the tools available in the standard package.

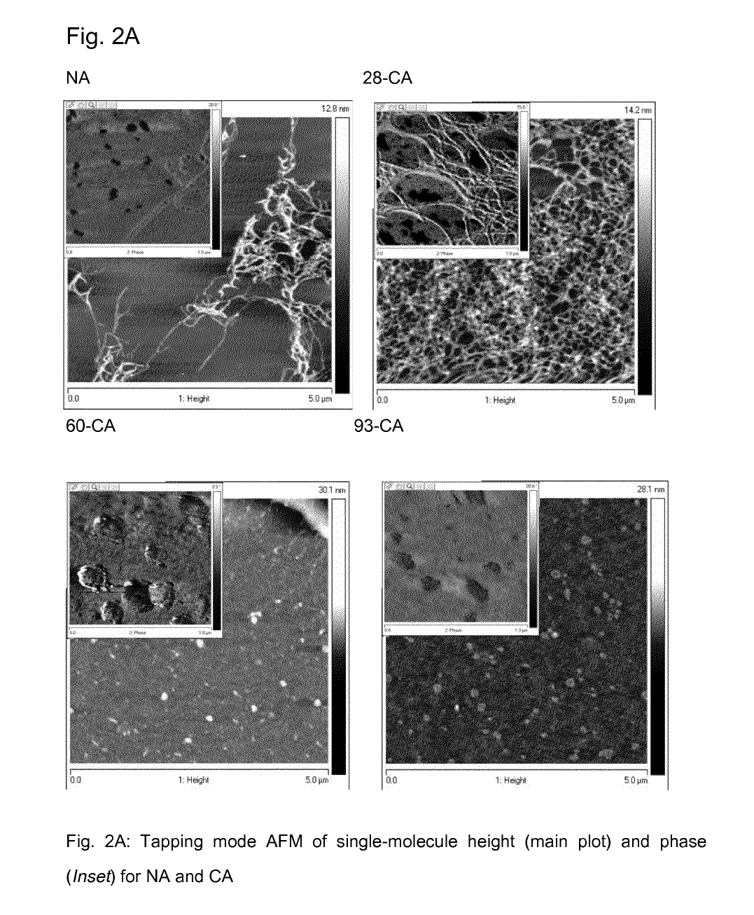

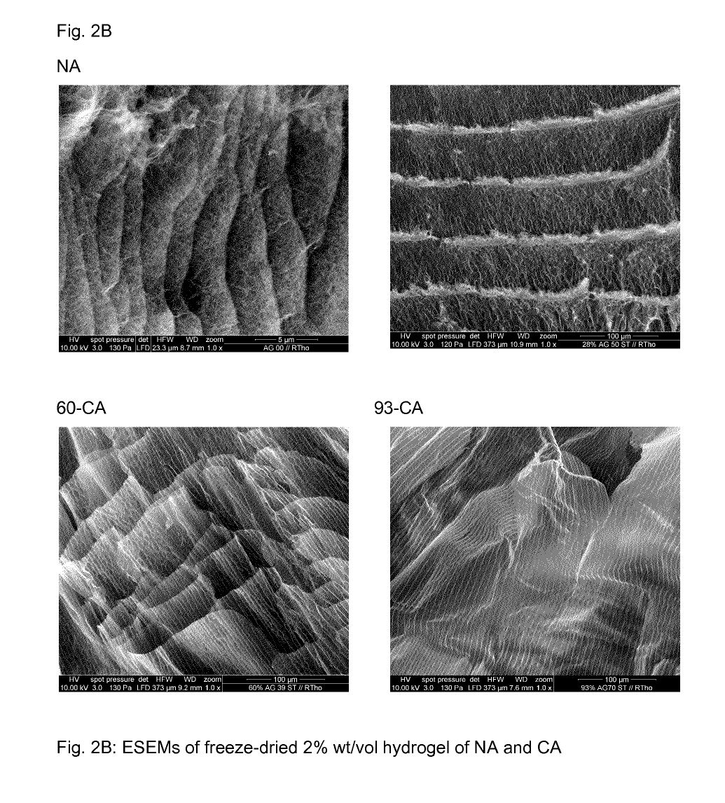

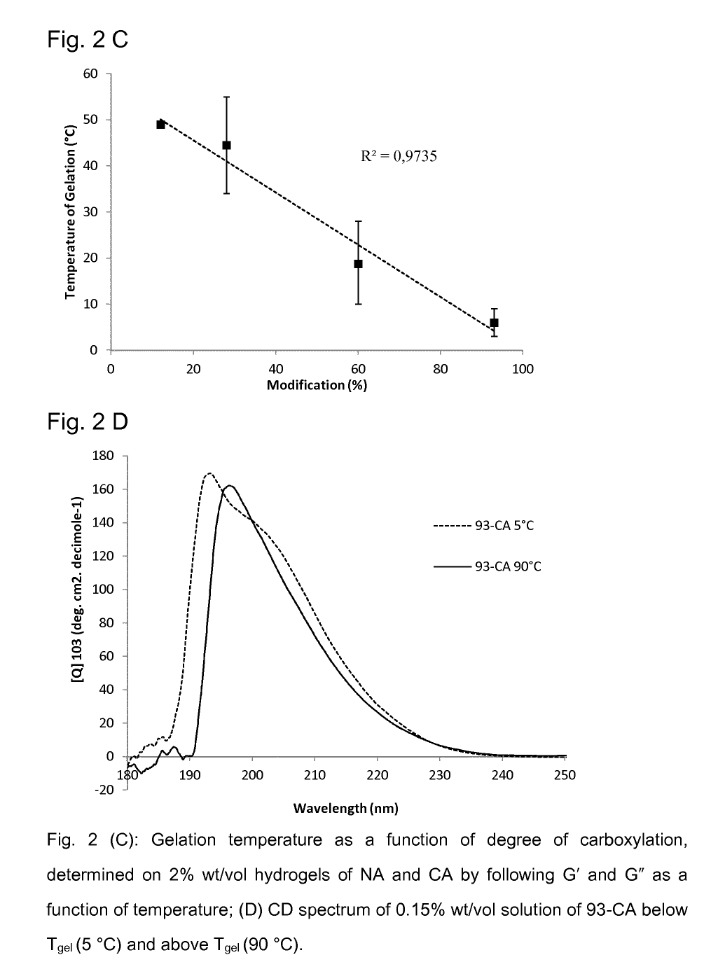

A technique commonly used to study secondary structure in biological molecules is circular dichroism (CD) (28). CD is very sensitive toward changes in the coupling of transition dipole moments, which serves as a probe for secondary structure, e.g., α-helices or β-sheets in proteins (28). In unmodified agarose the CD arises from coupling of C—O—C ether chromophores, leading to positive residual ellipticity with a maximum at 183 nm for α-helices (FIG. 2A) (29)). This ellipticity can be directly attributed to the α-helices, as it is absent in oligomeric agarose obtained from acid-catalyzed hydrolysis, which is incapable of organizing into an α-helix.

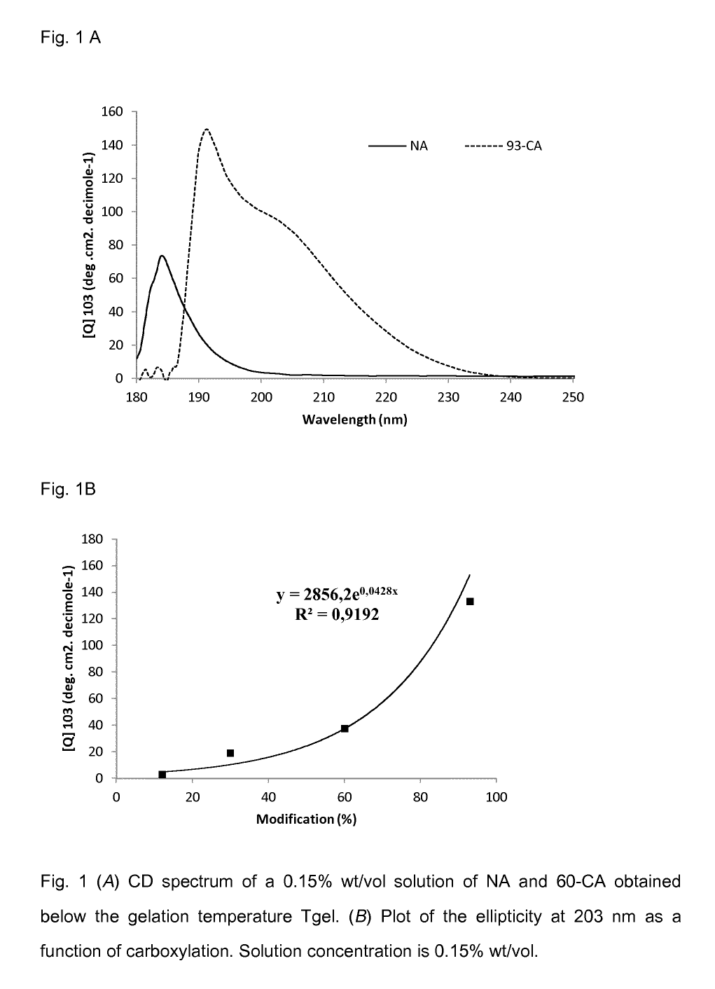

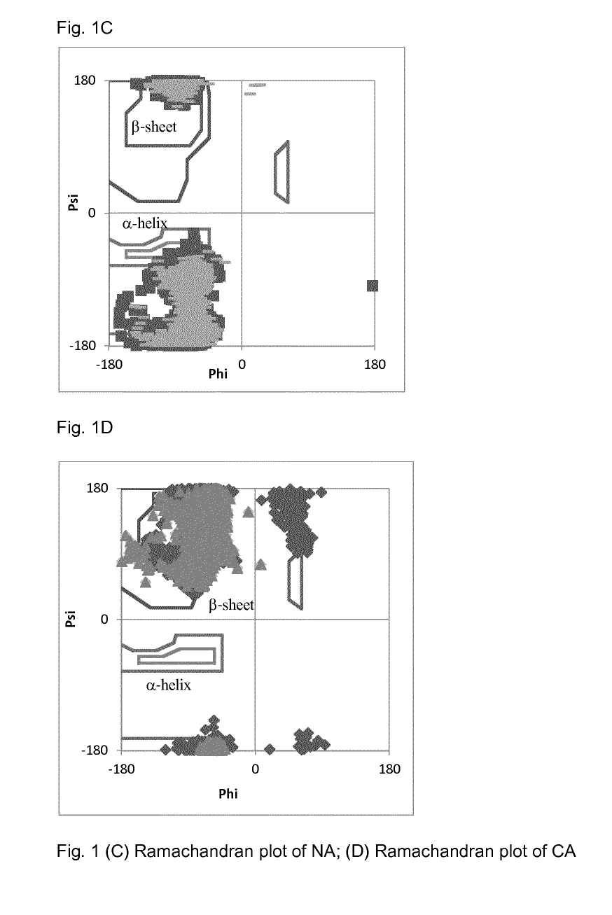

The changes to the agarose secondary structure upon carboxylation (to obtain carboxylated agarose, which will be referred to hereinafter as CA), can be seen by using 93% carboxylated agarose (93-CA) as a model system. Like unmodified agarose (natural agarose, referred to hereinafter as NA), 93-CA exhibits strong positive ellipticity; however, in comparison with NA the maximum is even stronger and red-shifted (191 nm) (FIG. 1A) and, additionally, the red shift is accompanied by the emergence of a new peak at 203 nm. The new ellipticity at 203 nm can be attributed to the carboxylation of the backbone, as its maximum increases exponentially with carboxylation (FIG. 1B). The change in molar absorptivity and shift to a lower energy excitation wavelength of the primary ellipticity may also be due to chromophore contributions of the introduced carboxyl group to the network of dipolar couplings in the a-helices in 93-CA. It therefore appears that the modification of the NA backbone promotes a reorganization of the chains leading to a new secondary structure in CA in addition to the native α-helices. In protein CD spectra positive ellipticity around 217 nm indicates β-sheets (28). Likewise, the secondary structure-related ellipticity at 203 nm in the CD spectrum of 93-CA may be attributed to a β-sheet-like conformation of the polysaccharide chains. Further evidence for the molecular reorganization leading to a new secondary structure can be obtained by analyzing the molecular dynamics (MD) simulation data. In proteins, the occurrence of helical or β-sheet motifs can be determined using the empirical Ramachandran plot (30). Extending this approach to polysaccharides (31), the empirical distributions of the dihedral angles φ and ψ of the glycosidic backbone were plotted (FIG. 1C). As expected, in the case of NA the Ramachandran plot reveals the predominance of helical conformation. However, in contrast the coordinates of the CA dihedrals are mainly located in the β-sheet region consistent with the CD spectra, and this implies a dramatic reorganization of the polysaccharide backbone upon carboxylation. Such an α-helix to β-sheet transformation, although reported in proteins, is highly restricted and has not been observed before in polysaccharides.

To ascertain the impact of the β-sheet structure on the organization of agarose molecules, tapping mode atomic force microscopy (AFM) (32) was used to visualize NA and carboxylated agarose (CA) molecules (FIG. 2A). It is clear that the NA strands are organized as helical structures (FIG. 2A, Inset), appearing like “a string of pearls”(FIG. 2A, Left). At 28% carboxylation (28-CA), the helical organization appears slightly disrupted and this is consistent with the CD data for 28-CA, where only a small shoulder associated with the ellipticity at 203 nm is observed. However, increasing carboxylation (60%, 60-CA; and 93%, 93-CA) results in the complete reorganization of fibers into disk-shaped structures that appear to have some residual helical motifs. Fibers of soluble amyloid-β (Aβ) peptide fibrils that possess mixed β-sheet structures, also form circular globules, like those observed in the 60-CA and the 93-CA (33, 34). Because the molecular mass of agarose after oxidation [Mₙ, 88-94 kDa; polydispersity index (PDI), 2.14-2.23] is virtually identical to that of NA (Mₙ, 95 kDa; PDI, 2.99-3.12), its contribution to the observed structural changes can be ruled out. The visual evidence is consistent with the CD data and the Ramachandran plot predictions, and is proof for the presence of a unique secondary structure in CA.

Without being bound to any theory, one could postulate a prominent role for reduced H bonding and increased electrostatic repulsion between chains upon carboxylation, which, in sum, may promote more hydrophobic interactions leading to hitherto unknown interactions between agarose molecules.

In proteins, changes to secondary structure can alter protein folding (tertiary structure) in a manner that favors aggregation. In fact, soluble Aβ has a disordered structure; however, aggregates of Aβ have a significant amount of β-sheet structure (35). If the paradigm for structure evolution is conserved between polysaccharides and proteins, then one might expect that the switch from a-helix to β-sheet could also impact the supramolecular assembly of the agarose molecules and hence the microstructure of the gel. To determine whether this indeed occurs and to what extent, the interior of freeze-dried 2% wt/vol hydrogels was characterized using environmental scanning electron microscopy (ESEM). Whereas the microstructure of the NA gel was composed of tufts of disordered fibers, microstructures of the CA gel bear no resemblance to NA and reveal an astonishing transformation in the organization of agarose fibers with an increasing degree of carboxylation (FIG. 2B). Even at a low degree of carboxylation (28%), the fibers are organized into ridge-like structures, composed of high-aspect ratio cells that appear to have some periodicity. Increasing the carboxylation to 60% further enhances this organization, wherein disk-shaped motifs appear to fuse to one another in columnar strands organized into lamellae. At 93% carboxylation, the fiber organization appears to have undergone a fundamental change, resulting in sheet-like structures composed of highly oriented ribbons. Because 93-CA chains are organized into disk-shaped structures, their assembly into sheets would require an unraveling followed by lateral stacking. It has been shown that the transformation of Aβ42 disk-shaped oligomers into fibrils involves organization of the peptide strands within these oligomers into β-sheets (36). The switching of the three-dimensional (3D) structure of a polymer hydrogel from a random organization of fibers to a lamellar structure has not been described before and has been found in the course of this invention.

The gelation of NA involves association of α-helices through H bonding mediated by the C6 primary hydroxyl group. Because carboxylation modifies helical interactions, it also imposes changes to the physicochemical characteristics of CA gels. The gelation behavior of agarose shows a hysteresis in that the melting temperature of the gel (Tₘ, >80 ° C.) is significantly higher than the gelation temperature (Tgel ˜40° C.) (23). This is expected, as the formation of the gel requires H bonding, which is more likely to occur as the entropy of the system is reduced. A key prediction of the MD simulation is that CA chains have markedly diminished associative tendencies, resulting in lower H-bond formation. One implication is a decrease of the gelation temperature, as promotion of H bonding requires lower kinetic energy. In fact, the complete carboxylation of agarose results in the lowering of the Tgel to below 10° C. (i.e., δ=−30° C.) over agarose, with intermediate carboxylation yielding intermediate T_{gel} (FIG. 2C).

The lower gelation temperature is advantageous for cell encapsulation and tissue regeneration applications, as activation of heat-shock proteins can be avoided (37).

Concrete proof for the direct involvement of the β-sheet in the gelation of the CA gels was obtained by following the CD spectrum of 93-CA (fully carboxylated agarose) as a function of temperature. The CD spectrum of the 93-CA gel above its Tₘ shows a complete abolishment of the ellipticity at 203 nm associated with the β-sheet structure and additionally a further red shift of the ellipticity at 199 nm in comparison with the gel at 5° C. (FIG. 2D). This is strong evidence that in CA the new β-sheet organization is responsible not only for the physical cross-linking of the gel but also for the dominant associative interaction between the polysaccharide chains.

Without being bound to any theory, the results obtained would be in accordance with a mechanism for the gelation of CA involving four steps of: (i) reorganization of the polymer backbone due to disruption of helices, resulting in a-helix to β-sheet switch; (ii) followed by aggregation of polymer chains through β-sheet motifs; (iii) elongation of these aggregates into high-aspect ratio structures; and (iv) the assembly of these high-aspect ratio structures in higher lamellar sheets.

Cross-links are often described as knots or entanglements of two and more chains. Because the gelation in both NA and CA can be attributed to association of secondary structure of a specific conformation, the cross-links can be imagined as assimilation of these secondary structures into soft spheres, and its formation can be linked to the growth of nano-particles through phase inversion. These highly specific associative processes can manifest in dilute solutions as aggregates. The size, polydispersity (PD), and zeta potential (ζ) of aggregates that are spontaneously formed in dilute solutions of NA and CA were determined using dynamic light scattering. The average size of aggregates formed in NA solution (0.15% wt/vol) was 1.09 μm, with a PD of ˜0.6, suggesting a rather heterogeneous associative process. In contrast, the aggregates formed from CA solutions were almost half the size, around 600 nm, and more narrowly dispersed (PD ˜0.3), implying a higher homogeneity. In fact, the changes to the size of the cross-links manifest themselves as a loss of turbidity in the gels, which is concomitant with increased carboxylation.

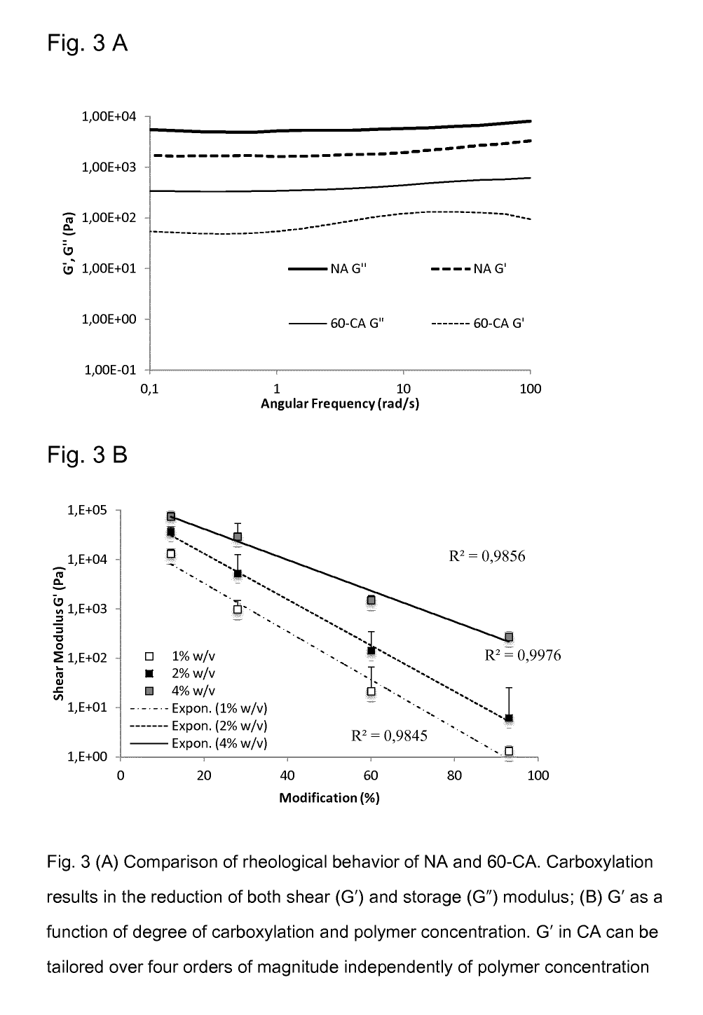

As these aggregates represent the origins of physical cross-linking, their charge characteristics influence their crystallization into a gel network. Although aggregates of NA have only a slightly negative ζ (−5 mV), ζ of the CA aggregate becomes increasingly negative (maximum −27 mV). Rheology studies reveal that increasing surface charge favors a more highly organized structure (38) but a much looser association due to electrostatic repulsion (39), thereby resulting in gels with a very well-defined microstructure, but which are physically weaker. CA gels have lower G′ and G″in comparison with NA. A typical rheology curve of storage (G′) and loss (G″) modulus as a function of angular frequency for NA and 60-CA is shown in FIG. 3A. The reduction in G′ is consistent with the predictions from the MD simulations, as a lower tendency for H bonding coupled with an increased charged density along the polysaccharide lowers friction at the molecular level, thereby reducing the shear modulus of the gel (39). More importantly, by varying the degree of carboxylation, the G′ of the hydrogels in accordance with the present invention can be tailored independent of the polysaccharide concentration, for example for a 2% wt/vol gel over four orders of magnitude (from 3.6×10⁴ Pa to 6 Pa), spanning the entire range of soft tissues found in the mammalian anatomy (40), and over a slightly reduced range of G′ for a 4% wt/vol gel (FIG. 3B).

The reduction of the shear modulus G′ of hydrogels based on disaccharides by a process wherein the hydrogel is subjected to a partial oxidation of the primary hydroxyl groups of the disaccharides constitutes a further embodiment of the present invention.

The origin of the changes to G′ can be exclusively attributed to the new secondary structure as both NA and CA have identical molecular weights and PDI.

The ability to influence secondary structure of polysaccharides via carboxylation is not limited to agarose but can also be demonstrated in other polysaccharides. κ-carrageenan, a polysaccharide, like agarose, also organizes into helical structures. Carboxylation of the primary alcohol at C6 position of sulfated D-galactose in κ-carrageenan results in changes to the CD spectra that are identical to those in agarose. Upon carboxylation, the negative residual ellipticity of κ-carrageenan β-helices undergoes a red shift from 183 nm to 189 nm, which is again accompanied by a new residual ellipticity with a maximum at 203 nm. The opposite sign of these two spectral features clearly demonstrates the co-existence of two secondary structural elements, β-helix and β-sheet, in a single polysaccharide. Furthermore, AFM images reveal that carboxylation of κ-carrageenan promotes the transition of polymer fibers from helical to disk-shaped assemblies as observed in CA. Even more remarkable is that the switch from helices to β-sheets, like in the case of agarose, induces reorganization of the microstructure of the freeze-dried gels from fibrous (unmodified κ-carrageenan, KC) to a high-ordered lamellar structure (carboxylated κ-carrageenan, CKC). The organization of polymer fibers in carboxylated κ-carrageenan is also driven by the association of β-sheets, like in CA, as can be seen from a lower modulus for carboxylated κ-carrageenan in comparison with κ-carrageenan. The G′ for carboxylated κ-carrageenan is three orders of magnitude lower than that for κ-carrageenan.

This shows that carboxylation of primary hydroxyl groups in hydrogels based on disaccharides provides a general approach for altering the secondary structure of α-helical polysaccharides.

CA60 Gels Promote Human Umbilical Vein Endothelial Cell (HUVEC) Organization into Lumens. The organization of cells into tissue-like structures involves a complex interplay between the soluble signals and those originating from the ECM (matrix stiffness, cell-ECM binding motifs, bound growth factors). Because stiffness of a biomaterial has been shown to impact stem cell lineage choices (1) and the metastasis of cancer cells (41), it can be expected that the injectable CA gels with tunable mechanical and structural properties in accordance with the present invention will be highly desirable for cell delivery and as a clinically translatable system for controlled tissue morphogenesis.

Vascularization is critical for the survival of cells and necessary for the transport of signaling molecules to aid in regeneration. Vasculogenesis, as it pertains to in vitro studies, is the formation of lumens from dispersed endothelial cells (ECs), and it differs from angiogenesis where endothelial structures form from an already existing blood vessel or an EC monolayer (42). It is well established that during vascular lumen morphogenesis, i.e., the formation of arteriole-like structures, cell-cell contacts and mural (support) cells play a vital role. To identify the factors that influence EC organization, several in vitro models have been established, including collagen gel, fibrin gel, and matrigel (43-46). These studies have revealed that arginine-glycine-aspartic acid (RGD) integrin binding sequence and soluble signals such as vascular endothelial growth factor (VEGF) and fibroblast growth factor-2 (FGF-2) are essential and that the mechanical aspects of the gel impact the formation of EC networks (43-46).

Nevertheless, the factors that impact the organization of multiple ECs in freestanding tubular structures are not fully understood. Because vasculogenesis primarily occurs during embryonic development when ECM is immature, a screening of the impact of the gel modulus and bound and soluble signals on human umbilical vein endothelial cell (HUVEC) organization was performed to evaluate the role of the immediate cellular environment in how soluble and bound signals are perceived by ECs. To investigate this premise further the organization of HUVECs in CA gels of two moduli, 0.02 kPa (CA60) and 1 kPa (CA28) was studied by systematically altering three parameters: basement membrane proteins (±0.01% wt/vol Matrigel), cell-binding motif (±RGD), and soluble signals [±VEGF, FGF, and Phorbol 12-myristate 13-acetate (PMA)]. In comparison, HUVECs were also cultured in fibrin gel and collagen gel supplemented with 0.01% Matrigel and soluble signals and in Matrigel supplemented with soluble signals.

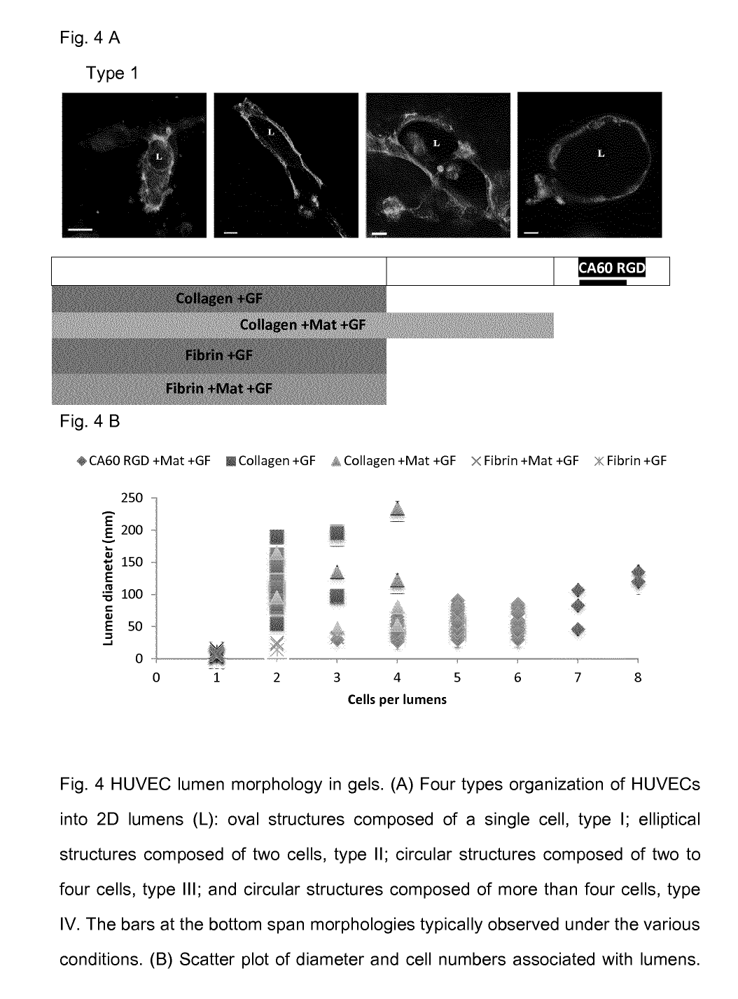

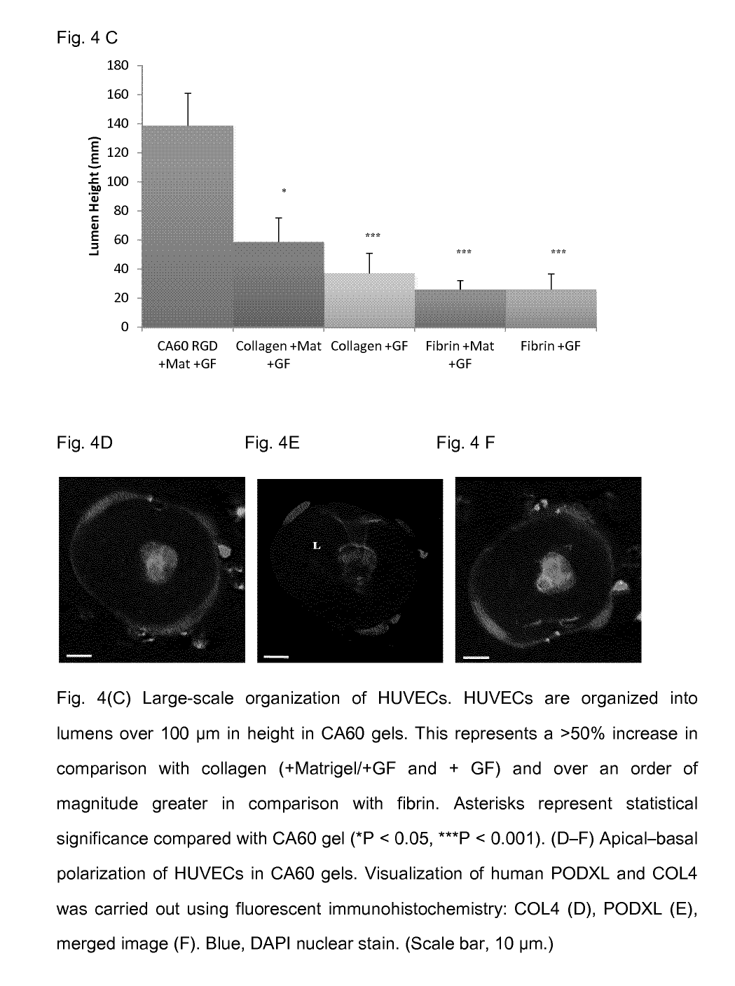

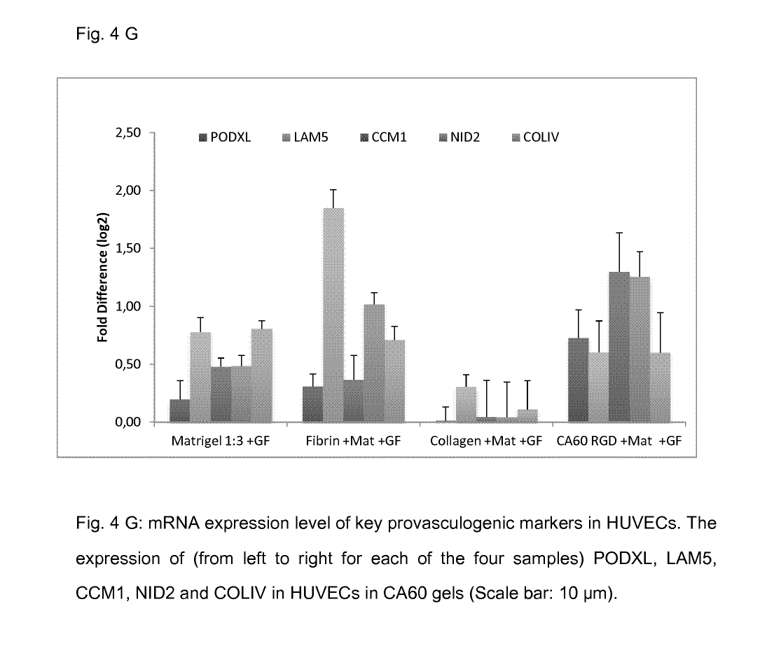

The organization of HUVECs into lumens can be categorized into four types as shown in FIG. 4A. In general, the organization of HUVECs in fibrin and collagen gels involved one to two cells and exhibited characteristics of type I and type II lumens (FIG. 4B). However, in contrast, HUVECs in CA60 modified with RGD and supplemented with basement membrane proteins and soluble signals showed type III and type IV structures, with more than three HUVECs participating in the formation of the lumens (FIG. 4B). Interestingly, no such organization was observed in the series of experiments for CA60 gels in the absence of RGD, basement membrane proteins, and soluble factors. Analysis of the frequency and structural characteristics (diameter and length) of the lumens revealed significant differences. In general more lumens were observed in CA60 gels in comparison with both fibrin and collagen gels (FIG. 4B). Furthermore, despite higher cell numbers per lumen the diameters of lumens formed in the CA60 gels were quite homogeneous at around 50-100 μm. However, more heterogeneity was observed in collagen and fibrin gels (FIG. 4B). Another significant observation was that HUVECs in CA60 gels could organize into freestanding, hollow, tubular structures over 100 μm in length, resembling arterioles (FIG. 4C). In contrast, the average length of such structures was about 50% smaller in collagen gels and an order of magnitude lower in fibrin gels (FIG. 4C). This may be attributed to the observed differences in the polarization potential of HUVECs as discussed below.

Apical-basal polarization of ECs is a critical step in the formation of stable blood vessels (47). Immunofluorescent staining against human podocalyxin (PODXL) and type-4 collagen (COL4A1) revealed apical and basal localization of PODXL and COL4A1, respectively, in HUVECs in CA60 gels, suggesting that they had undergone apical-basal polarization (FIG. 4D and E). In comparison, HUVECs in fibrin and collagen gels did not stain for human PODXL and COL4A1. This is consistent with the down-regulation at the mRNA level of PODXL and NID2, both of which are necessary for lumen expansion and maturation, in both collagen and fibrin gels in comparison with CA60 (FIG. 4G). It is noteworthy that the lumens appear to originate from a cluster of HUVECs that are already polarized, i.e., show apical localization of PODXL. This is in accordance with literature reports that vascular lumen morphogenesis requires the polarization of an EC cluster (three to five cells), which involves the recruitment of PODXL at the apical surface, which then initiates lumen expansion (47). A factor that might contribute to the formation of HUVEC clusters is the superior proliferation of the HUVECs in the CA60-RGD-modified gel, which is twofold greater than under expansion conditions on tissue culture plastic. Interestingly, the incorporation of basement membrane proteins, i.e., Matrigel, in the series of experiments made, had no effect on lumen length in fibrin gels and provided only a marginal increase in collagen gels (FIG. 4C) and this was also consistent with the lack of appreciable changes to the expression of key vasculogenesis markers at the mRNA level. This implies that the observed organization of HUVECs into lumens in CA60 gels cannot be attributed solely to the presence of growth factors and basement membrane proteins because HUVECs in Matrigel while staining positive for PODXL and COL4 do not show apical-basal localization and also do not organize into lumens. A noteworthy observation is that HUVECs in the CA28 gels, although showing comparable expression levels of the provasculogenic markers in comparison with HUVECs in CA60 gels at the mRNA level, however, remain dispersed and fail to organize, thus suggesting a role for biophysical variables.

Several mechanisms might contribute to the provasculogenic characteristics of the low-modulus CA gels. In biological gels such as fibrin and collagen, proteolytic degradation of the matrix by membrane-type matrix metalloproteinases is necessary and critical, as it paves the way for the migration and organization of the ECs (43). CA without additional factors or components cannot undergo proteolytic degradation, but could be slowly degraded through hydrolytic dissolution and possibly other as yet not completely understood mechanisms. At any rate, these are slow processes that take place at a much longer time-scale (weeks) than endothelial cell organization (1-2 days). Therefore, the role of matrix degradation in the organization of HUVECs in CA gels can be ruled out. However, it is possible that the stiffness and the chemistry of the gel can modulate the organization and affinity of ECM components and soluble signals. Because the stiffness of fibrin and collagen gels is similar to that of CA60, this suggests that the origin of the provasculogenic nature of CA60 may lie in its unique physicochemical properties (backbone charge and secondary structure) and not solely in its stiffness. Another aspect worth considering is the modulation of HUVEC function by the CA gel through a mechanobiology paradigm. This would require mechanical coupling between the gel matrix and the cell through the RGD motif and a unique role for this motif in HUVEC function. It has long been recognized that RGD signaling is important for EC survival and proliferation (48). Furthermore, it is well known that β1-integrin signaling is important in arterial tubulogenesis and β1 integrins have a binding site for RGD (49). Therefore, it is conceivable that β1 integrins on the HUVECs (50) mechanically couple to the gel through the RGD ligand and thereby sense the mechanical environment provided by the gel. Interestingly, among 32 conditions screened, HUVEC organization into lumens occurs only in CA60 gels modified with RGD. On the basis of these findings, it can be concluded that CA gels are well suited for understanding and leveraging the role of mechanobiology in tissue morphogenesis and provide a potential translational platform for regenerative therapies.

In this invention, injectable gels for Therapeutic Angiogenesis are disclosed. In particular gels comprising of carboxylated agarose of various degrees of carboxylation (CAXX, where XX denotes the degree of carboxylation from 10-100), optionally covalently modified with cell adhesion motifs such as the integrin binding sequence arginine-glycine-aspartic acid (RGD) and additionally containing VEGF, phorbol myristate acetate, and basement membrane proteins laminin, collagen type IV and entactin are disclosed. These gel formulations can induce morphologically accurate and physiological functional blood vessels with appropriate branching structures in vivo.

The following Examples show the hydrogels in accordance with the invention and their use.

### EXAMPLES

Modified agarose was obtained as follows:

Agarose type I has been obtained from Calbiochem. TEMPO (((2,2,6,6-Tetramethylpiperidin-1-yl)oxyl), NaOCl, NaBH4, NaBr, EDC (1-ethyl-3-(3-dimethylaminopropyl) carbodiimide)), MES buffer (2-(N-morpholino)-ethanesulfonic acid) have been obtained from Sigma Aldrich and used as received. Solution of 0.5 M NaOH have been freshly made every three months as well as solution of 5 M HCl. Peptide GGGGRGDSP has been obtained from Peptide International. Ethanol technical grade was used without any further purification. De-ionized water was used for non-sterile synthesis.

Agarose was modified under sterile conditions: All the chemicals were dissolved in autoclaved water and filtered with a 0.2 μm filter. All the glassware was autoclaved and the reaction was conducted under a laminar flow. Agarose (1 g) was autoclaved in MilliQ water. Autoclaved agarose was poured into a 3-necked round bottom flask. A mechanical stirrer was adapted to one of the necks. A pH-meter was adapted on the round bottom flask. The reactor was then cooled down to 0-5° C. and vigorously stirred. TEMPO (0.160 mmol, 20.6 mg) was added, NaBr (0.9 mmol, 0.1 g) and NaOCl (2.5 ml, 15% solution) was as well poured inside the reactor. The solution was adjusted to pH=10.8 with HCI and NaOH. The pH was maintained at 10.8 by adding NaOH. At the end of the reaction NaBH₄ (0.1 g) was added and pH=8 was reached. The solution was stirred for 1 hour and NaCl (0.2 mol, 12 g) and ethanol (500 ml) was added. The agarose was precipitated and extracted in a funnel. The two layers were then filtered on a frit glass. The agarose was then dialyzed in Spectra Pore 4, MWCO=12-14000 for 2 days and the water was changed two times. Prior to dialysis the membranes were left overnight in a 70% ethanol solution, 2 hours before use they were rinsed in autoclaved water. Finally the product was put on a Christ LD 2-8 LD plus at 0.1 mbar for the main drying and at 0.001 mbar during the desorption phase. Samples were put in round bottle flask and frozen in liquid nitrogen bath on a rotary evaporator modified for this purpose. Thin layers of frozen solution were obtained on the flask wall reducing the lyophilization time.

Five different hydrogels were prepared as follows:
NA—Hydrogel of unmodified Natural Agarose
CA60—soft hydrogel, (no RGD)
CA28—hard hydrogel (no RGD)
28RGD—CA28 covalently modified with RGD
6ORGD—CA60 covalently modified with RGD

NA stands for unmodified agarose, CA28 for agarose in which 28% of the primary hydroxyl groups are converted to carboxyl groups and CA60 for an agarose where 60% of the primary hydroxyl groups have been converted into carboxyl groups.

Growth Factors (GF) were introduced into the hydrogels using Matrigel (Mat), obtaining seven hydrogel formulations as follows:

1 60RGD+Mat+GF

2 28RGD+Mat+GF

3 NA—unmodified control

4 6ORGD+Mat

5 28RGD+Mat

6 CA60+Mat+GF

7 CA28+Mat+GF

The different gels were preloaded inside insulin syringes (BD Bioscience) and were kept on ice until injection.

In vivo Intramuscular Injection

Mice were anaesthetized before injection. 50 μL of cold phosphate buffer solution (PBS) were injected in the Gastrocnemius (two injections per leg, i.e. 4 in total per animal). The cold PBS injection was used to cool down the injection site before the injection of the gel. Directly thereafter 50 μL of the gel formulations formulation was injected at the same injection point that was marked with a pen. Only one condition was injected per leg. The animals were left 2 weeks under normal diet and then sacrificed.

### Tissue Histology

Mice were anesthetized and tissues were fixed by vascular perfusion with 1% paraformaldehyde in PBS pH 7.4. Gastrocnemius muscles were harvested, embedded in OCT compound (CellPath, Newtown, Powys, UK), frozen in freezing isopentane, and cryosectioned. Sections of 25 μm in thickness were stained with the following primary antibodies and dilutions: rat monoclonal anti-mouse CD31 (clone MEC 13.3, BD Biosciences, Basel, Switzerland) at 1:100; mouse monoclonal anti-mouse α-SMA (clone 1A4, MP Biomedicals, Basel, Switzerland) at 1:400; rabbit polyclonal anti-NG2 (Chemicon International, Hampshire, UK) at 1:200. Fluorescently labeled secondary antibodies (Invitrogen, Basel, Switzerland) were used at 1:200.

### Intravascular Lectin Staining

Physiological perfusion of induced vessels was assessed by intravascular staining with a fluorescently labeled Lycopersicum esculentum (tomato) lectin or biotinylated Lycopersicon esculentum lectin (Vector Laboratories, Burlingame, Calif., USA) that binds the luminal surface of blood vessels. Biotinylated Lycopersicon esculentum lectin was detected with fluorescently labeled streptavidin (eBioscience, Vienna, Austria). Briefly, mice were anesthetized and lectin was injected intravenously (50 μl of a 2 mg/ml lectin solution per mouse) and allowed to circulate for 4 min before vascular perfusion of 1% PFA in PBS pH 7.4 for 3 min under 120 mm/Hg of pressure.

### Vessel Quantifications

Vessel diameters and vessel length density were measured in muscle frozen sections after staining for CD31 (marker for endothelial cells), NG2 (marker for pericyite cells) and SMA (marker for smooth muscle cells). Briefly, vessel diameters were measured by overlaying captured microscopic images with a square grid. Squares were selected randomly and the diameter of each vessel, if present, in the defined square was measured (in μm). At least 100 diameters were randomly quantified for each experimental condition. Vessel length density was measured in at least 15 representative fields per muscle tracing the total length of vessels in each field and dividing it by the area of the field, which was kept constant for all measurements and all experimental conditions (mm of vessel length/mm² of surface area). Vascular segment length was defined as the average length (in μm) of the linear vessel segments comprised between two branch points. It was also measured in the same analyzed microscopy fields by counting the number of branching points in the vascular network (n) and dividing the total vessel length by n+1. All analyses were performed using the Cell P imaging software (Olympus, Volketswil, Switzerland).

### Statistics

The significance of differences was assessed using analysis of variance (ANOVA) followed by the Sidak test for multiple comparisons (GraphPad Prism 6). p<0.05 was considered statistically significant.

The vessel growth induced by different compositions 2 weeks after implantation and the corresponding quantifications of vascular parameters were investigated. As expected, the unmodified Agar condition (NA) also contained vascular structures, since it is based on a fully biocompatible material. The CA28 and, more extensively, the CA60 modifications alone caused a loss of vascular ingrowth despite the combination with matrigel and GF, but this was restored to various extents by the addition of the RGD sequence. Analysis of vessel diameter showed a homogeneous distribution in the size range of normal capillaries for all conditions, without enlarged aberrant structures.

It is interesting to note that
a) the presence of matrigel did not improve the amount of vascular ingrowth;
b) the presence of matrigel even caused the formation of vascular networks that are less branched, as evidenced by a longer average segment length, and therefore have a less beneficial connectivity. This is of some importance, because proper metabolic function of newly induced vasculature requires an orderly branching so as to achieve an efficient distribution of blood perfusion within the tissue that should receive the nutrients and exchange waste products;
c) efficient vascular ingrowth occured even in the absence of growth factors, i.e. it is not necessary to include growth factor in the compositions; and
d) the 28RGD and 60RGD conditions without either matrigel or GF display the shortest segment length, i.e. the highest branching and connectivity of induced vascular networks.

The 28RGD and 60RGD conditions, without addition of either matrigel or GF, displayed the best examples of morphologically ideal vascular structures, with properly branched capillary networks, mature and associated with NG2-positive and SMA-negative pericytes.

Lectin perfusion experiments showed that already after 2 weeks practically all induced vessels were functionally connected with the general circulation, without major differences in any condition, suggesting that in all conditions a process of angiogenesis (growth of new vessels from pre-existing ones) takes place rather than vasculogenesis (de novo assembly of endothelial structures from progenitor cells).

In order to be therapeutically useful, newly induced vascular structures must be able to persist long-term. This important step is defined as vascular stabilization and has been shown to take place within the first 4 weeks after induction of new angiogenesis (51-53). Therefore, the stabilization afforded by the different compositions to newly induced vascular structures was investigated 7 weeks after hydrogel implantation in vivo.

The unmodified agar and 28RGD hydrogel conditions displayed low vessel length density (VLD, which indicates the amount of new vessels induced; the higher the value, the more new vessels are induced), with poorly branched (segment length; the lower the segment length the more branched the vessels are) and immature endothelial structures, scarcely associated with NG2+/SMA− pericytes, indicating a significant regression of the capillaries that were induced after 2 weeks. Presence of GF in the 28RGD condition allowed a better stabilization of induced vessels, which however remained poorly covered by pericytes and poorly branched (segment length).

In contrast, the 6ORGD hydrogel displayed very dense and highly branched capillary networks, which were also mature, i.e. associated with NG2-positive and SMA-negative pericytes. Quantification of vessel length density showed that this composition ensured complete stabilization of induced angiogenesis, with no vascular regression and even further network expansion compared with the 2-week time-point.

Remarkably the addition of GF did not provide any clear benefit in terms of either amount of new vessels or branching of the induced networks compared to 60RGD hydrogel alone.

Vessel diameters did not show any statistical significance among the different groups.

### CITED DOCUMENTS

1. Engler A J, Sen S, Sweeney H L, Discher D E (2006) Matrix elasticity directs stem cell lineage specification. Cell 126(4):677-689.
2. Lipski A M, Pino C J, Haselton F R, Chen I-W, Shastri V P (2008) The effect of silica nanoparticle-modified surfaces on cell morphology, cytoskeletal organization and function. Biomaterials 29(28):3836-3846.
3. Lutolf M P, Doyonnas R, Havenstrite K, Koleckar K, Blau H M (2009) Perturbation of single hematopoietic stem cell fates in artificial niches. Integr Biol 1(1):59-69.
4. Lutolf M P, Hubbell J A (2005) Synthetic biomaterials as instructive extracellular microenvironments for morphogenesis in tissue engineering. Nat Biotechnol 23(1): 47-55.
5. Shastri V P (2009) In vivo engineering of tissues: Biological considerations, challenges, strategies, and future directions. Adv Mater 21(32-33):3246-3254.
6. Shastri V P (2012) Delivering regeneration. Drug Deliv Transl Res 2:293-296.
7. Elisseeff J, et al. (1999) Transdermal photopolymerization for minimally invasive im-plantation. Proc Natl Acad Sci USA 96(6):3104-3107.
8. Emans P J, et al. (2010) Autologous engineering of cartilage. Proc Natl Acad Sci USA 107(8):3418-3423.
9. Stevens M M, et al. (2005) In vivo engineering of organs: The bone bioreactor. Proc Natl Acad Sci USA 102(32):11450-11455.
10. Aimetti A A, Machen A J, Anseth K S (2009) Poly(ethylene glycol) hydrogels formed by thiol-ene photopolymerization for enzyme-responsive protein delivery. Biomaterials 30(30):6048-6054.
11. Lutolf M P, Hubbell J A (2003) Synthesis and physicochemical characterization of end-linked poly(ethylene glycol)-co-peptide hydrogels formed by Michael-type addition. Biomacromolecules 4(3):713-722.
12. DeForest C A, Polizzotti B D, Anseth K S (2009) Sequential click reactions for synthe-sizing and patterning three-dimensional cell microenvironments. Nat Mater 8(8): 659-664.
13. Ehrbar M, et al. (2007) Enzymatic formation of modular cell-instructive fibrin analogs for tissue engineering. Biomaterials 28(26):3856-3866.
14. Augst A D, Kong H J, Mooney D J (2006) Alginate hydrogels as biomaterials. Macromol Biosci 6(8):623-633.
15. Huebsch N, et al. (2010) Harnessing traction-mediated manipulation of the cell/matrix interface to control stem-cell fate. Nat Mater 9(6):518-526.
16. Stevens M M, Qanadilo H F, Langer R, Prasad Shastri V (2004) A rapid-curing alginate gel system: Utility in periosteum-derived cartilage tissue engineering. Biomaterials 25(5):887-894.
17. Tamura H, Nagahama H, Tokura S (2006) Preparation of chitin hydrogel under mild conditions. Cellulose 13:357-364.
18. Hartgerink J D, Beniash E, Stupp S I (2002) Peptide-amphiphile nanofibers: A versatile scaffold for the preparation of self-assembling materials. Proc Natl Acad Sci USA 99(8):5133-5138.
19. Kopecek J, Yang J (2009) Peptide-directed self-assembly of hydrogels. Acta Biomater 5(3):805-816.
20. Fernández-Cassiío S, León-Mateos A, Sampedro F G, Oreja M T C (2007) Biocompatibility of agarose gel as a dermal filler: Histologic evaluation of subcutaneous implants. Plast Reconstr Surg 120(5):1161-1169.
21. Selmi T A, Neyret P, Verdonk P C M, Barnouin L (2007) Autologous chondrocyte transplantation in combination with an alginate-agarose based hydrogel. Tech Knee Surg 6:253-258.
22. Selmi T A, et al. (2008) Autologous chondrocyte implantation in a novel alginate-agarose hydrogel: Outcome at two years. J Bone Joint Surg Br 90(5):597-604.
23. Lahaye M, Rochas C (1991) Chemical structure and physico-chemical properties of agar. Hydrobiologia 221(1):137-148.
24. Arnott S, et al. (1974) The agarose double helix and its function in agarose gel structure. J Mol Biol 90(2):269-284.
25. Djabourov M, Clark A H, Rowlands D W, Ross-Murphy S B (1989) Small-angle x-ray scattering characterization of agarose sols and gels. Macromolecules 22(1):180-188.
26. Cao X, Shoichet M S (2002) Photoimmobilization of biomolecules within a 3-dimensional hydrogel matrix. J Biomater Sci Polym Ed 13(6):623-636.
27. Myer Y P (1969) The pH-induced helix-coil transition of poly-L-lysine and poly-L-glutamic acid and the 238-mu dichroic band. Macromolecules 1379:624-628.
28. Woody R W, Berova N, Polavarapu P L, Nakanishi K, eds (2012) Comprehensive Chiroptical Spectroscopy (Wiley, New York), pp 475-497.
29. Arndt E R, Stevens E S (1997) Anhydro sugar and linkage contributions to circular dichroism of agarose and carrageenan, with conformational implications. Carbohydr Res 303(1):73-78.
30. Ramachandran G N, Ramakrishnan C, Sasisekharan V (1963) Stereochemistry of polypeptide chain configurations. J Mol Biol 7:95-99.
31. Lütteke T, Frank M, von der Lieth C-W (2005) Carbohydrate Structure Suite (CSS): Analysis of carbohydrate 3D structures derived from the PDB. Nucleic Acids Res 33(Database issue):D242-D246.
32. Takahiro F (2010) Atomic force microscopy imaging of food polysaccharides. Measurement 16(1):1-12.
33. van denAkker C C, Engel M F M, Velikov K P, Bonn M, Koenderink G H (2011) Morphology and persistence length of amyloid fibrils are correlated to peptide molecular structure. J Am Chem Soc 133(45):18030-18033.
34. Ganesh S, Jayakumar R (2003) Structural transitions involved in a novel amyloid-like beta-sheet assemblage of tripeptide derivatives. Biopolymers 70(3):336-345.
35. Blackley H K, et al. (2000) In-situ atomic force microscopy study of beta-amyloid fi-brillization. J Mol Bio1298(5):833-840.
36. Ahmed M, et al. (2010) Structural conversion of neurotoxic amyloid-beta(1-42) oligomers to fibrils. Nat Struct Mol Biol 17(5):561-567.
37. Whitley D, Goldberg S P, Jordan W D (1999) Heat shock proteins: A review of the molecular chaperones. J Vasc Surg 29(4):748-751.
38. Rugge A, Tolbert S H (2002) Effect of electrostatic interactions on crystallization in binary colloidal films. Langmuir 18(18):7057-7065.
39. Okano T, Araki J, Masahisa W, Shigenori K (1999) Influence of surface charge on viscosity behavior of cellulose microcrystal suspension. J Wood Sci 45:258-261.
40. Reilly G C, Engler A J (2010) Intrinsic extracellular matrix properties regulate stem cell differentiation. J Biomech 43(1):55-62.
41. Paszek M J, et al. (2005) Tensional homeostasis and the malignant phenotype. Cancer Cell 8(3):241-254.
42. Vailhé B, Vittet D, Feige J J (2001) In vitro models of vasculogenesis and angiogenesis. Lab Invest 81:439-452.
43. Lafleur M A, Handsley M M, Knäuper V, Murphy G, Edwards D R (2002) Endothelial tubulogenesis within fibrin gels specifically requires the activity of membrane-type-matrix metalloproteinases (MT-MMPs). J Cell Sci 115(Pt 17):3427-3438.
44. Sieminski A L, Hebbel R P, Gooch K J (2004) The relative magnitudes of endothelial force generation and matrix stiffness modulate capillary morphogenesis in vitro. Exp Cell Res 297(2):574-584.
45. Koh W, Stratman A N, Sacharidou A, Davis G E (2008) In vitro three dimensional collagen matrix models of endothelial lumen formation during vasculogenesis and angiogenesis. Methods Enzymol 443:83-101.
46. Arnaoutova I, George J, Kleinman H K, Benton G (2009) The endothelial cell tube formation assay on basement membrane turns 20: State of the science and the art. Angiogenesis 12(3):267-274.
47. Herbert S P, Stainier D Y R (2011) Molecular control of endothelial cell behaviour during blood vessel morphogenesis. Nat Rev Mol Cell Biol 12(9):551-564.
48. Oguey D, George P W, Ruegg C (2000) Disruption of integrin-dependent adhesion and survival of endothelial cells by recombinant adenovirus expressing isolated beta in-tegrin cytoplasmic domains. Gene Ther 7(15):1292-1303.
49. Shiokawa S, et al. (1999) Functional role of arg-gly-asp (RGD)-binding sites on β1 integrin in embryo implantation using mouse blastocysts and human decidua. Biol Reprod 60(6):1468-1474.
50. Leavesley D I, Schwartz M A, Rosenfeld M, Cheresh D A (1993) Integrin beta 1- and beta 3-mediated endothelial cell migration is triggered through distinct signaling mechanisms. J Cell Biol 121(1):163-170.
51. Dor Y, Djonov V, Abramovitch R, Itin A, Fishman G I, Carmeliet P, Goelman G, Keshet E. Conditional switching of vegf provides new insights into adult neovascularization and pro-angiogenic therapy. EMBO J. 2002;21:1939-1947.
52. Ozawa C R, Banfi A, Glazer N L, Thurston G, Springer M L, Kraft P E, McDonald D M, Blau H M. Microenvironmental vegf concentration, not total dose, determines a threshold between normal and aberrant angiogenesis. J Clin Invest. 2004;113:516-527.
53. Tafuro S, Ayuso E, Zacchigna S, Zentilin L, Moimas S, Dore F, Giacca M. Inducible adeno-associated virus vectors promote functional angiogenesis in adult organisms via regulated vascular endothelial growth factor expression. Cardiovasc Res. 2009;83:663-671.

1. Injectable hydrogels comprising polysaccharides based on disaccharides the backbones of which form an a-helix structure and in which in at least 10% of the disaccharide units the primary hydroxyl groups are oxidized.

2. Injectable hydrogels in accordance with claim 1 wherein in at least 20% of the disaccharide units the primary hydroxyl groups are oxidized.

3. Injectable hydrogels in accordance with claim 1 or 2 wherein in at least 35% of the disaccharide units the primary hydroxyl groups are oxidized.

4. Injectable hydrogels in accordance with claim 1 or 2 wherein in 50 to 95% of the disaccharide units the primary hydroxyl groups are oxidized.

5. Injectable hydrogels in accordance with claim 1 or 2 wherein in 55 to 93% of the disaccharide units the primary hydroxyl groups are oxidized.

6. Injectable hydrogels in accordance with any of claims 1 to 5 wherein the polysaccharide is selected from the group consisting of agarose and κ-carrageenan.

7. Injectable hydrogels in accordance with any of claims 1 to 6 wherein the polysaccharide is modified with cell adhesion motifs such as the integrin binding sequence arginine-glycine-aspartic acid (RGD), or with peptide sequences.

8. Injectable hydrogels in accordance with claim 7 wherein the peptide sequence is selected from YIGSR, IKVAV, MNYYSNS or PHSRN.

9. Injectable hydrogels in accordance with any of claims 1 to 8 comprising soluble signals.

10. Injectable hydrogels in accordance with claim 9 wherein the soluble signal is selected from vascular endothelial growth factor (VEGF), phorbol 12 myristate acetate (PMA), fibroblast growth factors (FGF), insulin growth factors (IGF), transforming growth factor beta-1(TGF-β) or platelet derived growth factor (PDGF).

11. Injectable hydrogels in accordance with any of claims 1 to 10 comprising components of the extracellular matrix (ECM).

12. Injectable hydrogels in accordance with claim 11 wherein the components of the extracellular matrix are selected from basement membrane proteins (BMP).

13. Injectable hydrogels in accordance with claim 12 wherein the basement membrane protein is selected from collagen type 4 (Col4), laminins (LAM) or entactin (also known as nidogen) or mixtures thereof.

14. Use of the injectable hydrogels in accordance with any of claims 1 to 13 in therapeutic angiogenesis.

15. A method for reducing the shear modulus G′ of hydrogels of polysaccharides based on disaccharides the backbones of which form an α-helical structure wherein the hydrogels are subjected to oxidation of the primary hydroxyl groups of the disaccharide units.

16. A process for inducing new vasculature in tissue comprising the use of injectable hydrogels in accordance with any of claims 1 to 13.
